(19) United States
(12) Patent Application Publication
(10) Pub. No.: US 2025/0281635 A1
(43) Pub. Date: Sep. 11, 2025

(54) EXTRACELLULAR VESICLE COMPOSITES AND THEIR USES IN THE TREATMENT OF LIVER INJURY

(71) Applicants: National Taiwan University, Taipei City (TW); Taipei Medical University, Taipei City (TW)

(72) Inventors: Thai-Yen LING, Taipei City (TW); Cheng-Chung LEE, New Taipei City (TW); Hwei-Jiung WANG, San Diego, CA (US); Hsin-Hung LIN, Taipei City (TW); Yen-Ting LU, Taipei City (TW)

(21) Appl. No.: 18/600,802

(22) Filed: Mar. 11, 2024

(51) Int. Cl. A61K 47/69; A61K 31/353; A61K 31/675; A61K 31/7072; A61K 31/708; A61K 38/21; A61K 39/00; A61P 1/16; C07K 16/28

(52) U.S. Cl. CPC A61K 47/6901; A61K 31/353; A61K 31/675; A61K 31/7072; A61K 31/708; A61K 38/21; A61P 1/16; C07K 16/2851; A61K 2039/505

(57) ABSTRACT

Disclosed herein are extracellular vesicle (EV) composites and their uses in the treatment of liver injury, particularly, acute liver failure (ALF). The EV composite includes an EV derived from a mesenchymal stem cell (MSC), and a recombinant polypeptide conjugated to the EV via a click chemistry reaction. Preferably, the recombinant polypeptide is a single-chain variable fragment (scFv) that recognizes and binds to asialoglycoprotein receptor 1 (ASGR1) or ASGR2. The present disclosure thus also encompasses a method of treating ALF in a subject. The method includes the step of administering an effective amount of the EV composite to the subject to alleviate symptoms associated with the ALF.

### SEQUENCE LISTING

The present specification refers to a Sequence Listing, which is submitted electronically as a.xml file named “MYHP_0219US_Sequence_Listing.xml”. The.xml file was generated on Mar. 7, 2024 and is 2 kilobytes in size. The entire contents of the Sequence Listing are herein incorporated by reference.

### BACKGROUND OF THE INVENTION

### 1. Field of the Invention

The present disclosure relates to extracellular vesicle composites, i.e., exosomes having recombinant polypeptides conjugated thereto. More particularly, the present disclosure relates to methods for treating liver injury (e.g., acute liver failure) by the use of said composites.

### 2. Description of Related Art

Acute liver failure (ALF) refers to the development of severe acute liver injury with impaired synthetic function and altered mental status in a patient without cirrhosis or preexisting liver disease. Because ALF often involves the rapid deterioration of mental status and the potential for multiorgan failure, patients should be managed in the intensive care unit at a transplant center. Early institution of medicament or specific therapy may prevent the need for liver transplantation and reduce the likelihood of poor outcomes.

Exosomes are membrane-bound extracellular vesicles (EVs) produced via inward budding from the endosomal compartment of most eukaryotic cells. EVs are found in biological fluids (e.g., saliva, urine, blood, etc) and are also released in vitro from cultured cells into their growth medium. EVs contain various molecular constituents of their cell-of-origin, including proteins and RNA, thus, EVs are important biomarkers in the diagnosis of diseases. Additionally, EV may also serve as drug delivery vesicles due to their ability to retain active molecules (e.g., proteins, DNA, drugs, etc) in their internal space (i.e., lumen) and transfer molecules from one cell to another via membrane vesicle trafficking.

The present study thus aims at providing an improved way of treating ALF, in which a therapeutic agent is delivered to a targeted treatment site with the aid of EVs, particularly, EVs that have been modified for targeted delivery of the therapeutic agent.

### SUMMARY

In one aspect, the present disclosure is directed to an extracellular vesicle (EV) composite, which comprises an EV derived from a mesenchymal stem cell (MSC); and a recombinant polypeptide conjugated to the EV via a click reaction.

According to embodiments of the present disclosure, the recombinant polypeptide has an amino acid sequence of SEQ ID No: 1.

According to embodiments of the present disclosure, the EV or the recombinant polypeptide is modified to comprise an azide, an alkene, an alkyne, a tetrazine, or a strained alkyne, provided that when the EV is modified to comprise the azide, then the recombinant polypeptide is modified to comprise the alkyne or the strained alkyne, or vice versa; or when the EV or the MSC is modified to comprise the tetrazine, then the recombinant polypeptide is modified to comprise the alkene, or vice versa.

In some embodiments, the EV is derived from the MSC treated with N-azidoacetyl-mannosamine thereby conferring the EV to comprise an azide on its outer surface, while the recombinant polypeptide is treated with a molecule having a click chemistry group that is dibenzocyclooctyne (DBCO), difluorinated cyclooctyne (DIFO), biarylazacyclooctynone (BARAC) or bicyclononyne (BCN) thereby conferring the recombinant polypeptide to comprise a strained alkyne, and the recombinant polypeptide is conjugated to the EV via strain promoted azide-alkyne cycloaddition (SPAAC). Preferably, the recombinant polypeptide is treated with a molecule having a DBCO group.

In other embodiments, the EV is derived from the MSC treated with 1,2,3,4-tetrazine, 1,2,3,5-tetrazine, or 1,2,4,5-tetrazine thereby conferring the EV to comprise a tetrazine on its outer surface, while the recombinant polypeptide is treated with a molecule having a click chemistry group that is oxanorbornadiene or cyclooctene thereby conferring the recombinant polypeptide to comprise an alkene, and the recombinant polypeptide is conjugated to the EV via the alkene and tetrazine inverse-demand Diels-Alder reaction.

Examples of the MSC suitable for use in the present disclosure include but are not limited to, adipose-derived MSC (AD-MSC), amniotic fluid-derived MSC (AF-MSC), bone marrow-derived MSC (BM-MSC), dental pulp-derived MSC (DP-MSC), endometrial-derived MSC (En-MSC), fallopian tube-derived MSC (FT-MSC), gingival-derived MSC (G-MSC), periodontal ligament-derived MSC (PDL-MSC), peripheral blood-derived MSC (PB-MSC), placenta-derived MSC (PD-MSC), placenta choriodecidua-derived MSC (PC-MSC), skeletal muscle-derived MSC (Sk-MSC), synovial membrane-derived MSC (SM-MSC), umbilical cord-derived MSC (UCB-MSC), and Wharton's Jelly-derived MSC (WJ-MSC). Preferably, the MSC is PC-MSC.

In another aspect, the present disclosure aims to provide a method of treating acute liver failure in a subject. The method includes the step of administering an effective amount of the EV composite of the present disclosure to the subject to alleviate symptoms associated with acute liver failure.

According to embodiments of the present disclosure, the EV composite is administered to the subject in the amount of about 1×10⁸ to 1×10¹² EV composite/mL.

According to optional embodiments of the present disclosure, the method further includes the step of administering an additional therapeutic agent to the subject for the treatment of acute liver failure. Examples of the therapeutic agents suitable for use with the EV composite of the present disclosure include but are not limited to, interferon, adefovir, entecavir, lamivudine, telbivudine, tenofovir, and silymarin.

In all embodiments, the subject is a human.

Many of the attendant features and advantages of the present disclosure will become better understood regarding the following detailed description considered in connection with the accompanying drawings.

### BRIEF DESCRIPTION OF THE DRAWINGS

The present description will be better understood from the following detailed description read in light of the accompanying drawings, where:

FIG. 1. Characterization of pcMSCs-EVs. (A) Nanoparticle tracking analysis (NTA): The size of pcMSCs-EVs isolated from conditioned medium (1 mL) of pcMSCs ranged from 100 to 150 nm with a concentration of 10⁹ particles/ml; (B) Zetasizer: The zeta potential and polydispersity index (PDI) of pcMSCs-EVs were measured by NanoZS/ZEN3600 Zetasizer (Malvern Instruments, UK); (C) Nanoparticle Flow cytometry: pcMSCs-EVs were found to have high levels of the tetraspanins CD9, CD63, and CD81 in their membrane, as measured by NanoFCM.

FIG. 2. pcMSCs-based therapies protect C3A cells from acetaminophen (APAP) injury in vitro. (A) (B) (C) APAP dose-dependently reduced the viability of C3A cells, while the viability was significantly improved after 48 hours of pcMSCs-conditioned medium (pcMSCs-CM) incubation in the presence of APAP (10 mM) as determined by luminescent cell viability assay. (D) (E) APAP reduces GSH expression in a dose-dependent manner, while GSH levels significantly increased after 48 hours of incubation with pcMSCs-CM in the presence of APAP (10 mM), as measured by a GSH Assay Kit. (F) (G) The viability of C3A cells was significantly improved after 48 hours of pcMSCs-EVs (10⁹ particles) incubation in the presence of APAP (10 mM). Three replicates per sample were performed, and the experiment was repeated three times. The data are presented as mean±SEM. *P<0.05, **P<0.01, ***P<0.001, ****P<0.0001.

FIGS. 3A to 3C. pcMSCs-based therapies attenuate APAP-induced liver injury in the ALF model. (A) Serum levels of alanine aminotransferase (ALT) and aspartate aminotransferase (AST) of sham group mice (n=13) or ALF mice treated with PBS (control) (n=8), pcMSCs (n=6), pcMSCs-CM (n=8) or pcMSCs-EVs (n=8) were measured at 24 hrs after saline or APAP injection; (B) Serum levels of inflammatory cytokines including IL-1β, IL-6, TNF-α, and MCP-1 were measured after pcMSCs, pcMSCs-CM or pcMSCs-EVs administrations (n=5-7 per group); (C) The quantified results of liver necrosis area of sham group mice or APAP-treated mice treated with PBS (control), pcMSCs, pcMSCs-CM or pcMSCs-EVs measured at 24 hrs after saline or APAP injection (n=5 per group). The data are presented as mean±SEM. *P<0.05, **P<0.01, ***P<0.001, ****P<0.0001.

FIG. 4. Optimization of cell-surface labeling of azido glycans on pcMSCs & pcMSCs-EVs via glycoengineering and click chemistry. (A) Azide groups were dose-dependently expressed on pcMSCs surface via glycoengineering while pcMSCs were supplemented with increasing concentrations of Ac4ManNAz for 3 days; (B) The highest expression level of azide groups on pcMSCs surface was observed at 4 days of Ac4ManNAz (20 μM) incubation; (C) The viability of Ac4ManNAz-treated pcMSCs was analyzed by luminescent cell viability assay; and (D) Azide groups distributed on pcMSCs-EVs surface dose-dependently conjugated with increasing doses of AZDye 488 DBCO through click reaction; The data are presented as mean±SEM;

FIG. 5. Targeting efficiency of scFv to ASGR1. (A) Sandwich ELISA was performed to detect serial dilutions of scFv that bound to ASGR1. The scFv was then detected using an HRP-conjugated anti-6× His tag antibody, and the absorbance at 450 nm was quantified using a microplate reader designed for fluorescence measurements. Three replicates per sample were performed, and the experiment was repeated three times. The data are presented as mean±SEM. (B) Different mol fractions of galactose to scFv were mixed and added to the wells containing ASGR1 antigen. The signal of scFv was dose-dependently inhibited by increasing concentrations of galactose. Three replicates per sample were performed, and the experiment was repeated three times. The data are presented as mean±SEM.

FIG. 6. Targeting efficiency of scFv-DBCO to ASGR1. Sandwich ELISA was performed to detect serial dilutions of scFv-DBCO that bound to ASGR1. scFv-DBCO was detected with Biotin-PEG3-azide (10 μM) via click reaction and subsequently interacted with streptavidin HRP and TMB. The absorbance was detected at 450 nm measured by a microplate fluorescence reader. Three replicates per sample were performed. The data are presented as mean±SEM.

FIGS. 7A to 7D. Characterization of TG-EVs. (A) Nanoparticle tracking analysis (NTA): Both Crtl-EVs and TG-EVs had a size ranging from 30 to 150 nm with a concentration of 10⁹ particles/mL; (B) Zetasizer: The zeta potential of Ctrl-EVs and TG-EVs were measured by NanoZS/ZEN3600 Zetasizer (C) Targeting efficiency: HepG2/C3A cells treated with increasing dose (10⁷, 10⁸, 10⁹) of TG-EVs for 18 hours of incubation exhibited intense Alexa Fluor-488 and Cy5 fluorescent signal in a dose-dependent manner compared to the cells treated with corresponding doses of Ctrl-EVs. (D) Therapeutic efficacy: TG-EVs significantly improved the viability of HepG2/C3A cells dose-dependently compared to corresponding doses of Ctrl-EVs after 48 hours of incubation in the presence of APAP (10 mM). Three replicates per sample were performed, and the experiment was repeated three times. The data are presented as mean±SEM. *P<0.05, **P<0.01, ***P<0.001, ****P<0.0001.

FIG. 8. TG-EVs exhibited superior therapeutic efficacy in attenuating APAP-induced liver injury in the ALF mouse model compared to Ctrl-EVs. (A) Serum levels of alanine aminotransferase (ALT) and aspartate aminotransferase (AST) of sham group mice (n=13) or APAP-treated mice administered PBS (control) (n=8), pcMSCs (n=6), pcMSC-CM (n=8), Ctrl-EVs or TG-EVs (n=5-8) were measured at 24 hours after saline or APAP injection. (B) Serum levels of inflammatory cytokines, such as IL-1β, IL-6, TNF-α, and MCP-1, were measured after pcMSCs, pcMSC-CM, Ctrl-EV, or TG-EV administrations (n=5-7 per group). (C) Quantification of H&E staining was performed for liver sections from each group. The mean necrotic area (expressed as a percentage) per field was calculated from a total of six fields from four liver samples in each group. The data are presented as mean±SEM. *P<0.05, **P<0.01, ***P<0.001, ****P<0.0001.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

For convenience, certain terms employed in the specification, examples, and appended claims are collected here. Unless defined otherwise, all technical and scientific terms used herein have the same meaning as commonly understood by one of the ordinary skilled in the art to which this invention belongs.

### 1. Definitions

As used herein, the term “extracellular vesicle” or “EV” refers to a cell-derived vesicle comprising a membrane that encloses an internal space. EV comprises all membrane-bound vesicles that have a smaller diameter than the cell from which they are derived. EV generally ranges in diameter from 20 nm to 1,000 nm. Examples of EV include but are not limited to, fragments of cells, vesicles derived from cells by direct or indirect manipulation (e.g., by serial extrusion or centrifugation), and vesicles produced by living cells (e.g., by direct plasma membrane budding), etc. EV can be derived from a living or dead organism, explanted tissues or organs, or primary and/or cultivated cells. According to preferred embodiments of the present disclosure, EV is derived from primary cells or producing cells (e.g., mesenchymal stem cells (MSCs)). Additionally or optionally, the EV isolated from the producing cells (e.g., MSCs) may be treated with a therapeutic agent so that the therapeutic agent is coupled to the membrane of the EV and/or encapsulated in the lumen of the EV.

The terms “administration”, “administering,” and grammatical variants thereof refer to introducing a composition, e.g., an EV or an EV composite of the present disclosure, into a subject via a pharmaceutically acceptable route. The introduction of a composition (e.g., the EV composite) of the present disclosure into a subject is by any suitable route, including orally, intranasally, parenterally (intravenously, intra-arterially, intramuscularly, intraperitoneally or subcutaneously), rectally, intralymphatically, intrathecally, periocularly or topically. A suitable route of administration allows the composition or the agent to perform its intended function.

The term “modified,” when used in the context of an EV, a mesenchymal stem cell (MSC), or a polypeptide described herein, refers to an alteration or engineering of the EV, MSC, or polypeptide such that the modified EV, MSC or polypeptide independently comprises a click chemistry group, provided that the click chemistry group on the modified EV or MSC is complementary to the click chemistry group on the modified polypeptide in a Cu-free click reaction. Examples of the click chemistry group include an azide, an alkene, an alkyne, a tetrazine, and a strained alkyne. In some embodiments, the EV or the MSC is modified to comprise the azide, and the recombinant polypeptide is modified to comprise the alkyne or the strained alkyne. In other embodiments, the EV or the MSC is modified to comprise the tetrazine, and the recombinant polypeptide is modified to comprise the alkene.

The term “a recombinant polypeptide” or “a recombinant protein” as used herein refers to a polypeptide or a protein produced via recombinant technology. Recombinantly produced polypeptides and proteins expressed in engineered host cells are considered isolated for the present disclosure, as are native or recombinant polypeptides which have been separated, fractionated, or partially or substantially purified by any suitable technique. The polypeptide disclosed herein can be recombinantly produced using methods known in the art. Alternatively, the proteins and polypeptides can be chemically synthesized. In some aspects of the present disclosure, recombinant polypeptides conjugated to EVs are recombinantly produced by overexpressing the polypeptides in host cells, the overexpressed polypeptides are then purified from the host cells, and subsequently modified to comprise a click chemistry group, which is complementary to the click chemistry group present on the EV derived from MSCs. In some embodiments, the recombinant polypeptides are scFvs that specifically recognize asialoglycoprotein receptor 1 (ASGR1). In other embodiments, the recombinant polypeptides are scFvs that specifically recognize ASGR2.

As discussed herein, minor variations in the amino acid sequences of polypeptides are contemplated as being encompassed by the presently disclosed and claimed inventive concept(s), providing that the amino acid sequence maintain at least 70% identical after variation, such as at least 70%, 71%, 72%, 73%, 75%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% identical after variation. The present polypeptide may be modified specifically to alter a feature of the polypeptide unrelated to its physiological activity. For example, certain amino acids can be changed and/or deleted without affecting the physiological activity of the polypeptide in this study (i.e., its ability to target liver cells and treat liver injury). In particular, conservative amino acid replacements are contemplated. Conservative replacements are those that take place within a family of amino acids that are related in their side chains. Whether an amino acid change results in a functional peptide can readily be determined by assaying the specific activity of the peptide derivative. Fragments or analogs of proteins/polypeptides can be readily prepared by those of ordinary skill in the art.

The term “treatment” as used herein is intended to mean obtaining a desired pharmacological and/or physiologic effect, e.g., ameliorating liver injury. The effect may be prophylactic in terms of completely or partially preventing a disease or symptom thereof and/or therapeutic in terms of a partial or complete cure for a disease and/or adverse effects attributable to the disease. “Treatment” as used herein includes preventative (e.g., prophylactic), curative or palliative treatment of a disease in a mammal, particularly human; and includes (1) preventative (e.g., prophylactic), curative or palliative treatment of a disease or condition (e.g., liver injury) from occurring in an individual who may be pre-disposed to the disease but has not yet been diagnosed as having it; (2) inhibiting a disease (e.g., by arresting its development); or (3) relieving a disease (e.g., reducing symptoms associated with the disease).

The term “an effective amount” as used herein refers to an amount effective, at dosages, and for periods necessary, to achieve the desired result concerning the treatment of a disease. For example, in the treatment of a liver injury, an agent (i.e., the EV composite of the present disclosure) that decreases, prevents, delays, suppresses or arrests any symptoms of the liver injury would be effective. Similarly, in the treatment of a condition in need of tissue repair or regeneration, an agent (i.e., the EV composite of the present disclosure) that decreases, prevents, delays, suppresses, or arrests any symptoms of the condition or promotes liver tissue repair or regeneration would be effective. An effective amount of an agent is not required to cure a disease or condition but will provide treatment for a disease or condition such that the onset of the disease or condition is delayed, hindered, prevented, or the disease or condition symptoms are ameliorated. The effective amount may be divided into one, two, or more doses in a suitable form to be administered one, two, or more times throughout a designated period.

The term “subject” or “patient” is used interchangeably herein and is intended to mean a mammal including the human species that is treatable by the EV composite and/or method of the present invention. The term “mammal” refers to all members of the class Mammalia, including humans, primates, domestic and farm animals, such as rabbits, pigs, sheep, and cattle; as well as zoo, sports, or pet animals; and rodents, such as mouse and rat. Further, the term “subject” or “patient” is intended to refer to both the male and female gender unless one gender is specifically indicated. Accordingly, the term “subject” or “patient” comprises any mammal that may benefit from the treatment method of the present disclosure. Examples of a “subject” or “patient” include but are not limited to, a human, rat, mouse, guinea pig, monkey, pig, goat, cow, horse, dog, cat, bird, and fowl. In an exemplary embodiment, the patient is a human. Ranges of values are disclosed herein. The ranges set out a lower limit value and an upper limit value. Unless otherwise stated, the ranges include all values to the magnitude of the smallest values (either lower limit value or upper limit value) and ranges between the values of the stated ranges.

The singular forms “a”, “and”, and “the” are used herein to include plural referents unless the context dictates otherwise.

### 2. EV Composites

Aspects of the present disclosure pertain to EV composites, their production, and their uses in the treatment of liver injury. In one aspect, the present disclosure pertains to a novel EV composite, which comprises in its structure, an EV derived from a mesenchymal stem cell (MSC); and a recombinant polypeptide conjugated to the EV via a click reaction.

To join the recombinant polypeptide and the EV together, both the EV and the recombinant polypeptide are respectively modified to comprise complementary click chemistry groups before actual conjugation occurs. The term “complementary” as used herein in the context of click chemistry group refers to two chemical groups that may react to form a covalent bond therebetween in a click reaction, such as the pair of azide and alkyne/strained alkyne groups, the pair of tetrazine and alkene groups, and the like. According to embodiments of the present disclosure, each of the complementary click chemistry groups may be independently selected from the group consisting of an azide, an alkene, an alkyne, a tetrazine, and a strained alkyne, provided that when the EV or the MSC is modified to comprise the azide, then the recombinant polypeptide is modified to comprise the alkyne or the strained alkyne, or vice versa; or when the EV or the MSC is modified to comprise the tetrazine, then the recombinant polypeptide is modified to comprise the alkene, or vice versa. After such modification, the EV and the recombinant polypeptide are joined together via reactions between the two complementary click chemistry groups in a copper-free click reaction thereby forming the desired EV composite. Examples of the copper-free click reaction include but are not limited to, strain-promoted azide-alkyne cycloaddition (SPAAC), the alkene and tetrazine inverse-demand Diels-Alder reaction, and the like.

According to embodiments of the present disclosure, the EV is derived from MSC according to methods known to any skilled artisan in a related field. Preferably, the EV is harvested from the culture medium of MSC via ultracentrifugation. More preferably, the EV is isolated from the culture medium of MSC that has been pre-treated with a molecule having a click chemistry group thereby conferring the EV isolated therefrom also comprises the same click chemistry group. According to preferred embodiments of the present disclosure, the MSCs are pre-treated with N-azidoacetyl-mannosamine thereby conferring the EV isolated therefrom to comprise an azide on its outer surface.

Examples of the MSC suitable for use in the present disclosure include but are not limited to, adipose-derived MSC (AD-MSC), amniotic fluid-derived MSC (AF-MSC), bone marrow-derived MSC (BM-MSC), dental pulp-derived MSC (DP-MSC), endometrial-derived MSC (En-MSC), fallopian tube-derived MSC (FT-MSC), gingival-derived MSC (G-MSC), periodontal ligament-derived MSC (PDL-MSC), peripheral blood-derived MSC (PB-MSC), placenta-derived MSC (PD-MSC), placenta choriodecidua-derived MSC (PC-MSC), skeletal muscle-derived MSC (Sk-MSC), synovial membrane-derived MSC (SM-MSC), umbilical cord-derived MSC (UCB-MSC), and Wharton's Jelly-derived MSC (WJ-MSC). According to preferred embodiments of the present disclosure, the EV is derived from PC-MSC (i.e., isolated from the culture medium of PC-MSC).

Alternatively, or optionally, the MSCs may be pre-treated with an additional therapeutic agent before the harvest of EVs thereby generating EVs that include the additional therapeutic agents as a payload in the lumen. Accordingly, the additional therapeutic agents may be co-delivered to a treatment site along with the EV composites of the present disclosure. Examples of therapeutic agents suitable for treating producing cells in the present disclosure include but are not limited to, interferon, adefovir, entecavir, lamivudine, telbivudine, tenofovir, and silymarin.

Similar to the modification made to MSC or the EV derived therefrom, the present recombinant polypeptide is also modified to comprise a click chemistry group, which is complementary to the click chemistry group on the EV. According to embodiments of the present disclosure, the recombinant polypeptide may be a therapeutic polypeptide, such as an antibody or a single-chain variable fragment (scFv). According to embodiments of the present disclosure, the recombinant polypeptide is a scFv that recognizes and binds to asialoglycoprotein receptor 1 (ASGR1) or ASGR2. The ASGR1 or ASGR2 is known to facilitate liver infection by multiple liver viruses including hepatitis B, accordingly, ASGR1 or ASGR2 is also a target for liver-specific drug delivery. The antibody or the scFv may be produced via techniques well-known in the related art (e.g., recombinant gene engineering technology).

Various known techniques may be employed to modify the present recombinant polypeptide to comprise a click chemistry group. In some embodiments, the recombinant polypeptide is directly linked to the click chemistry group via a peptide linker. In such instances, the peptide linker may have a cysteine residue capable of forming a thioester bond with a maleimide group on a molecule having the click chemistry group; the peptide linker having the click chemistry group linked thereto is then connected to the recombinant polypeptide via the formation of an amide bond. Alternatively, the recombinant polypeptide may be linked to the click chemistry group via sortase-mediated ligation, in which the recombinant polypeptide is engineered to have a sortag expressed at its C-terminus, an enzyme sortase may then be used to facilitate the conjugation of the click chemistry group with the recombinant polypeptide by recognizing the sortag sequence. Alternatively, the recombinant polypeptide may be linked to the click chemistry group via site-specific mutation. In such strategy, the recombinant polypeptide is engineered to have a cysteine residue at its C-terminus, which allows the recombinant polypeptide to form a thioester bond with a maleimide group in the molecule having a click chemistry group thereby conferring the recombinant polypeptide to comprise the click chemistry group. According to preferred embodiments of the present disclosure, the recombinant polypeptide is a scFv that recognizes and binds to ASGR1 and has an amino acid sequence of SEQ ID No: 1, in which the C-terminus of the scFv of SEQ ID NO: 1 is modified with a cysteine mutation and a His-tag.

According to some embodiments of the present disclosure, the EV is derived from the MSC treated with N-azidoacetyl-mannosamine thereby conferring the EV to comprise an azide on its outer surface, while the recombinant polypeptide is treated with a molecule having a click chemistry group that is dibenzocyclooctyne (DBCO), difluorinated cyclooctyne (DIFO), biarylazacyclooctynone (BARAC) or bicyclononyne (BCN) thereby conferring the recombinant polypeptide to comprise a strained alkyne, and the recombinant polypeptide is conjugated to the EV via SPAAC. Preferably, the recombinant polypeptide has a cysteine residue at its C-terminus and is treated with a molecule having a DBCO group (e.g., DBCO-PEG4-maleimide).

According to some embodiments of the present disclosure, the EV is derived from the MSC treated with 1,2,3,4-tetrazine, 1,2,3,5-tetrazine, or 1,2,4,5-tetrazine thereby conferring the EV to comprise a tetrazine on its outer surface, while the recombinant polypeptide is treated with a molecule having a click chemistry group that is oxanorbornadiene or cyclooctene thereby conferring the recombinant polypeptide to comprise an alkene, and the recombinant polypeptide is conjugated to the EV via the alkene and tetrazine inverse-demand Diels-Alder reaction.

As the EV composite thus produced comprises the ASGR1-specific scFv, the EV composite may serve as a medicament for treating liver diseases (e.g., liver injury).

### 3. Uses of the Present EV Composites in the Treatment of Liver Injury

The present disclosure thus also encompasses a method of treating liver injury, particularly, acute liver failure (ALF) in a subject. The method includes the step of administering an effective amount of the EV composite of the present disclosure to a subject in need of such treatment so that symptoms associated with the liver injury (e.g., acute liver failure) are reduced and/or ameliorated.

According to embodiments of the present disclosure, the EV composite is administered to the subject in the amount of about 1×10⁸ to 1×10¹² EV composite/mL, such as 1×10⁸, 1×10⁹, 1×10¹⁰, 1×10^{11,} or 1×10¹² EV composite/mL. The EV composite may be administered to the subject via pharmaceutically acceptable route, preferably, via parenterally administration. The dose can be administered in a single aliquot, or alternatively in more than one aliquot. The skilled artisan or clinical practitioner may adjust the dosage or regime following the physical condition of the patient or the severity of the disease. The skilled artisan or health practitioner may adjust the dosing regimen of the present EV composite following various factors, such as age, gender, weight, and other treatments (if any). For example, the present EV composite may be administered to the subject 1-7 times per week (e.g., 1, 2, 3, 4, 5, 6, or 7 times per week) for 1, 2, 3, 4, or more consecutive weeks. Alternatively, the present EV composite may be administered to the subject 1-10 times (e.g., 1, 2, 3, 4, 5, 6, 7, 8, 9, or 10 times) every 2 weeks, every 4 weeks, every 5 weeks, every 6 weeks, every 7 weeks, every 8 weeks, every 9 weeks, or every 10 weeks; or once every month, every 2 months, or every 3 months, or longer. Preferably, the present EV composite is administered to the subject daily for at least 1 day, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more days. More preferably, the present EV composite is administered to the subject daily for at least 28 days (i.e., 4 weeks). Even more preferably, the present EV composite is administered to the subject daily for at least 14 days (i.e., 2 weeks). According to one working example of the present disclosure, the present EV composite is administered only once to the subject daily for the entire course of treatment (i.e., 2 days) to produce the therapeutic effect.

Additionally, or optionally, an additional therapeutic agent may be administered before, concurrently with, or after the administration of the present EV composite to the subject for the treatment of ALF. Examples of the therapeutic agents suitable for use with the EV composite of the present disclosure include but are not limited to, interferon, adefovir, entecavir, lamivudine, telbivudine, tenofovir, and silymarin.

The following Examples are provided to elucidate certain aspects of the present invention and to aid those skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLES

### Materials and Methods

Isolation and Characterization of Human Placenta Choriodecidua-Derived Mesenchymal Stromal Cells (pcMSCs)

Placenta-derived mesenchymal stromal cells (pcMSCs) were obtained from the human placentas' choriodecidual membrane. To isolate the cells, the tissues of the choriodecidual membrane from the placentas were digested using a combination of SMEM medium supplemented with 1 mg/mL DNase I, 0.5 mg/ml collagenase B, and 0.5 mg/ml protease. This digestion protocol was conducted overnight at 4° C., and the resultant mixture was filtered using a 100-μm nylon membrane. Following centrifugation, the cells were harvested and resuspended in a medium consisting of MCDB201 supplemented with 1% penicillin/streptomycin, 10 ng/ml epidermal growth factor, and 1% insulin transferrin selenium. Subsequently, the cells were seeded on culture plates coated with human collagen type IV. The adherent cells were maintained in the culture medium, with regular changes every 3 or 4 days to remove nonadherent cells. The cultured cells that demonstrated characteristics of placenta-derived MSCs were further cultured in a serum-free medium, exhibiting a fibroblast-like morphology after attachment. These cells showed positive expression for CD44, CD73, CD90, and CD29 while displaying negative expression for CD45, CD34, CD14, and HLA-DR.

### C3A (HepG2/C3A) Cell Culture

C3A (HepG2/C3A) was maintained in a 37° C., 5% CO₂, water-saturated atmosphere and grown in Dulbecco's modified Eagle medium (DMEM; Gibco BRL, Grand Island, NY, US) (Cat No. 31600034) supplemented with 1% (v/v) penicillin and streptomycin, 10% (v/v) FBS, 2 mM L-glutamine, 0.1 mM non-essential amino acid and sodium bicarbonate (3.7 g/L).

### Animals

Male C57BL/6 J mice, aged 8-12 weeks, were obtained from the National Laboratory Animal Center (NLAC) in Taipei, Taiwan (R. O. C.). They were housed in a specific pathogen-free (SPF) environment with unrestricted access to food and water. The experimental protocol was approved by the Institutional Animal Care and Use Committee (IACUC) in Taiwan, ensuring compliance with ethical guidelines for animal research.

### Acute Liver Failure (ALF) Mouse Model

Male C57BL/6 J mice were subjected to a fasting period of 12-16 hours, after which they were administered acetaminophen (APAP) (12 mg/ml) dissolved in a 0.9% NaCl solution via intraperitoneal injection. To optimize the APAP dosage, the fasted mice were given doses of 0, 50, 100, 150, 200, 250, and 300 mg/kg body weight of APAP. In the MSC treatment group, a total of 1×10⁷ pcMSCs were suspended in 1 ml of PBS and transplanted through intraperitoneal injection 30 minutes after administering 250 mg/kg body weight of APAP. In the MSC-CM treatment group, 1 ml of the cultured supernatant of pcMSCs was collected, and a single intraperitoneal injection was performed 30 minutes after administering 250 mg/kg body weight of APAP. For the sham and control groups, 1 ml of PBS was administered intraperitoneally 30 minutes after administering 0.9% NaCl solution and APAP, respectively. At 24 hours following APAP administration, mice were sacrificed, and serum samples were harvested for analysis.

### Extracellular Vesicles (EVs) Isolation and Characterization

EVs were obtained and purified via ultracentrifugation. Briefly, the conditioned medium from pcMSCs incubating for 4-10 days was collected, and centrifuged at 300×g for 10 minutes to eliminate dead cells. The resulting supernatant underwent sequential centrifugations conducted at 2000×g for 10 minutes to eliminate apoptotic bodies and 10,000×g for 30 minutes to remove microvesicles (MVs). Afterward, the EVs were purified through ultracentrifugation at a speed of 100,000×g for 90 minutes. To minimize the presence of soluble factors, the EVs were washed once with PBS. Following the removal of the supernatant, the EV pellets were collected and suspended in PBS. The BCA Protein Assay Kit was utilized to measure the protein content of the EVs. Nanoparticle tracking analysis (NTA) and the NanoZS/ZEN3600 Zetasizer (Malvern Instruments, UK) were employed to determine particle size, zeta potential, and particle concentration. The structure and shape of the EVs were observed using transmission electron microscopy (TEM). Nanoparticle flow cytometry (NanoFCM) was utilized to identify exosome-associated markers, including CD81, CD9, and CD63.

Cell-Surface Labeling of Azido Glycans With Fluorescent Probes on pcMSCs & pcMSCs-EVs by Glycoengineering and Click Chemistry

To find out the optimal concentration of N-azidoacetyl-mannosamine (Ac4ManNAz), pcMSCs were seeded in 96-well plates at a density of 3.4×10³ cells per well, and were subsequently exposed to different concentrations of Ac4ManNAz (0, 1, 5, 10, 20, 50, 100 μM) and incubated for a duration of 4 days. After removing the supernatant, the cells were washed twice with PBS and treated with AZDye 488 DBCO (20 μM) for 1 hour at 37° C. After undergoing five washes with PBS, the microplate reader was utilized to measure the fluorescence intensity (λex: 485 nm and λem: 528 nm). Similarly, the incubation time of Ac4ManNAz on pcMSCs was optimized by incubating the cells with 20 μM Ac4ManNAz for 1 to 6 days. AZDye 488 DBCO (20 μM) was then added for 1 hour, and each sample was quantified using a microplate fluorescence reader (Biotek). To evaluate the toxicity of Ac4ManNAz on pcMSCs, different concentrations of Ac4ManNAz (0, 1, 5, 10, 20, 50, 100 μM) were added to the cells and incubated for 4 days. The viability of each well was measured using the CellTiter-Glo® luminescent cell viability assay kit, and the luminescence was detected using a microplate reader (Biotek).

For the pcMSCs-EVs labeling experiment, pcMSCs were incubated with 20 μM Ac4ManNAz for 4 days, and EVs with azido glycans (N3-pcMSCs-Evs) were purified using ultracentrifugation as previously described. AZDye 488 DBCO (20 μM) was then added to N3-pcMSCs-Evs and incubated at 4° C. overnight. The final product was washed twice with PBS, and the fluorescence intensity of pcMSCs-EVs (λex: 485 nm and λem: 528 nm) was analyzed using a NanoAnalyzer (NanoFCM Co., Ltd., UK) with the assistance of NF Professional 1.0 software.

Conjugation of scFv-DBCO With Azido Glycans on pcMSCs or pcMSCs-EVs by Click Chemistries

To conjugate scFv-DBCO with azido glycans on pcMSCs or pcMSC-EVs, 10 μg of scFv-DBCO was prepared and incubated with 10⁶ N3-pcMSCs at 37° C. for 1 hour or 30 μg of N3-pcMSCs-EVs at 4° C. overnight. After incubation, the cells were washed with PBS three times, and the EVs were washed with PBS twice using ultracentrifugation.

Following conjugation, an ELISA was performed to validate scFv-EVs. First, each well of a 96-well ELISA plate was coated with recombinant anti-asialoglycoprotein receptor 1 antibody (2 μg/ml) diluted in coating buffer and incubated overnight at 4° C. Afterward, the plate was treated with 3% BSA to prevent nonspecific binding and subsequently exposed to ASGR1 human recombinant protein (2 μg/ml) for 1 hour. Next, various doses of scFv-EVs and ctrl-EVs (0, 50, 250, 1000, 2000 ng/ml) were added to the wells and incubated for 2 hours. After washing the plate with PBS five times, an anti-CD63 antibody conjugated with biotin (2 μg/ml) was used to detect EVs that bind with ASGR1. Following another wash to remove unbound antibody, streptavidin-HRP enzyme conjugates were added. A TMB substrate was then added to visualize the HRP, and the enzymatic reaction was halted using a stop solution. The optical absorbance at 450 nm was quantified using a microplate reader.

### Cell Confocal Imaging

C3A (HepG2/C3A) cells were plated on 12 mm microscope glass coverslips in 24-well plates at a concentration of 1.25×10⁵ cells per well. The cells were then fixed using a 4% PFA aqueous solution and blocked with 3% BSA in PBS. For the scFv internalization experiment, recombinant anti-asialoglycoprotein receptor 1 antibody (10 μg/ml) and scFv (10 μg/ml) were prepared using 1% BSA in PBS. In the galactose blocking group, scFv (10 μg/ml) was added after blocking with D-(+)-Galactose (100 mg/ml) for 1 hour. The secondary antibody used for the control and scFv groups was Alexa Fluor® 488 anti-His tag antibody, which was diluted in the blocking buffer at a ratio of 1:15000. For the recombinant anti-asialoglycoprotein receptor 1 antibody group, Alexa Fluor® 488-conjugated AffiniPure Donkey Anti-Rabbit IgG diluted in blocking buffer at a ratio of 1:200 was used. The slices were imaged using a Zeiss LSM780 confocal microscope system, and the obtained images were analyzed using ZEN5 microscopy software.

Membrane Labeling of DiR on pcMSCs-EVs

Firstly, pcMSCs were stained with 2.5μM DIR (DilC18 (7); 1,1′-Dioctadecyl-3,3,3′,3′-Tetramethylindotricarbocyanine lodide) at a temperature of 25° C. for 5 minutes.

The stained cells were then seeded in a 10-cm dish after being washed with PBS three times. Following three days of cell culture, the conditioned medium of the stained pcMSCs was collected for the purification of DiR-labeled EVs. The pellets containing the DiR-labeled EVs were subsequently resuspended in DPBS.

In Vitro Analysis of APAP-Treated C3A Cells With pcMSCs-Based Therapy

C3A (HepG2/C3A) cells were seeded in 96-well plates at a density of 1.5×10⁴ cells/well. For the viability assay, the cells were treated with different concentrations of APAP (0, 2, 5, 10, 20, and 40 mM) and incubated for 24 hours. After incubation, the cell viability was assessed using the CellTiter-Glo® luminescent cell viability assay kit, and luminescence was measured using a microplate reader (Biotek). To evaluate the therapeutic effects of pcMSCs-conditioned medium (pcMSCs-CM) and pcMSCs-EVs, an equal volume of pcMSCs-CM and various doses of pcMSCs-EVs (10⁷, 10⁸, 10⁹ particles/well) were added to each well after 30 minutes of incubation with 10 mM APAP. The viability of the treated cells was measured after 48 hours of pcMSCs-CM and pcMSCs-EVs incubation. To analyze apoptosis using Annexin V-FITC/PI double staining, C3A cells were seeded in 6-well plates at a density of 6.9×10⁵ cells/well. The cells were collected after trypsinization and centrifugation. The cell pellets were then resuspended in a binding buffer, and a staining solution comprising Annexin V-FITC and PI was introduced to the cell suspension in the dark for 15 minutes. The fluorescence was analyzed using a FACSAria II flow cytometer (BD Biosciences) with FlowJo™ Software (BD Bioscience). The level of GSH in C3A cells after pcMSCs-CM treatment was measured using a GSH assay kit.

scFv Production and Purification

The DNA fragment encoding the S. marcescens scFv was chemically synthesized and then inserted into the pET-21a(+) vector (Novagen) at the Ndel/BamHI sites. The protein sequence included a C-terminal cysteine mutation and a His-tag for conjugation and purification purposes. The codons were optimized for efficient expression in E. coli. Subsequently, the plasmid was transformed into E. coli BL21 (DE3) strain and the cells were cultured in Power Broth medium (Molecular Dimensions) supplemented with 0.1 mg/L ampicillin at 37° C. until the OD600 reached 1.5. The cell culture was then cooled to 16° C., and protein expression was induced by adding 1 mM isopropyl β-D-1-thiogalactopyranoside (IPTG). Following overnight incubation at 16° C., the cells were harvested by centrifugation, washed, and suspended in a buffer containing 500 mM NaCl and 50 mM Tris·HCl at pH 8.0. For the construction of the C-terminal cysteine mutant, the Quick-change site-directed mutagenesis kit (Stratagene) was employed. The wild-type plasmid served as the template, and the mutant was generated using the following pair of oligonucleotides. The mutant was expressed using the same methods as the wild type. The cells were disrupted using a Constant Systems cell disruptor, and the cell debris was removed by centrifugation. The supernatant was loaded onto a HisTrap Excel Ni-NTA column (GE Healthcare), washed with a buffer, and the proteins were stepwise eluted with 80-, 250-, and 500-mM imidazole. The 250 mM fraction contained the majority of the scFv protein and was concentrated to 15 mg/ml using Centricon (Millipore) or Jumbosep (PALL) with a 30 K membrane, while simultaneously exchanging the buffer to 100 mM NaCl and 50 mM Tris·HCl at pH 8.0.

scFv and DBCO-PEG4-Maleimide Conjugation by Site-Specific Cysteine-Cyclooctyne Reaction

The scFv was incubated with a 4-fold molar excess of CalFluor 488 Azide at 25° C. for 1 hour, followed by the addition of an equivalent of DBCO-PEG4-Maleimide in 50 mM HEPES (200 mM NaCl, pH 7.0), and incubated at 30° C. for 2 hours. The final product was analyzed by SDS-PAGE under a non-reducing condition.

### Statistical Analysis

The mean values of all experimental data were analyzed using GraphPad Prism 8.40 software. The two-tailed unpaired t-test was utilized to assess the level of statistical significance in variances between the experimental groups. Statistical significance was established by considering p-values below 0.05. All graphs depict mean values accompanied by the standard error of the mean (SEM).

### Example 1 Preparation and Characterization of pcMSCs-EVs

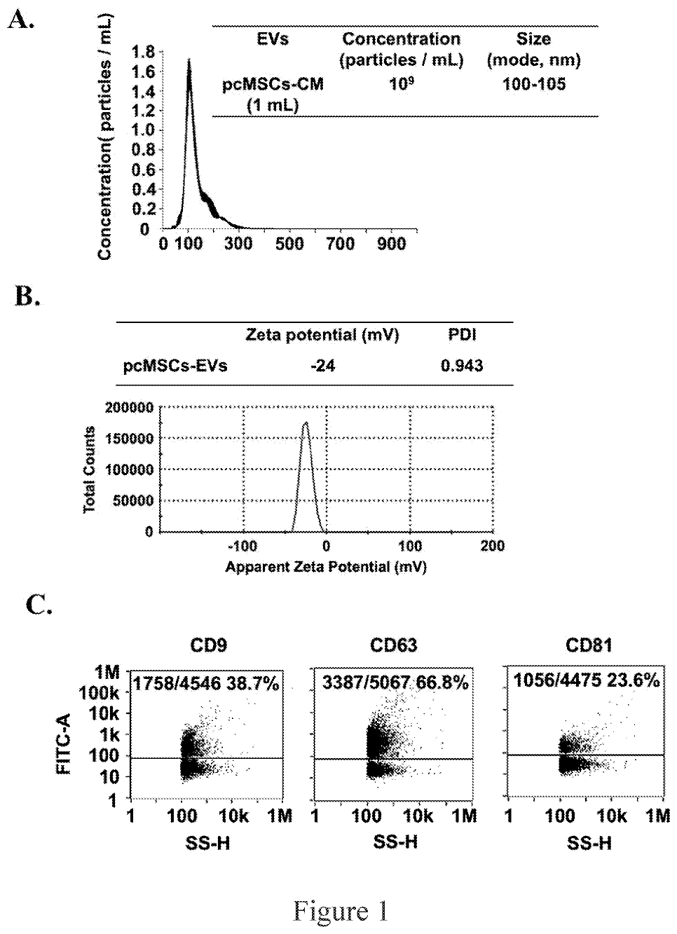

EVs were isolated from the conditioned medium of pcMSCs via ultracentrifugation by procedures described in the “Materials and methods” section and were termed “pcMSCs-EVs” hereafter. The pcMSCs-EVs were then subjected to morphology, size, and flow cytometry analyses. Results are provided in FIG. 1.

Transmission electron microscopy (TEM) analysis revealed that the pcMSCs-EVs predominantly exhibited a round or cup-shaped morphology, with diameters ranging from 30 to 150 nanometers (data not shown). Nanoparticle tracking analysis (NTA) was then conducted to analyze the size and concentration of pcMSCs-EVs. The results indicated that the size of pcMSCs-EVs ranged from 100 to 150 nm, with particle concentrations of roughly 10⁹ particles/mL (FIG. 1, (A)). Further characterization using Zetasizer involved measuring the zeta potential and polydispersity index (PDI) of pcMSCs-EVs. Zeta potential (ZP) is a quantitative measurement of the electrical charge and stability of particles suspended in a fluid. It provides insight into the particle-particle interactions and the propensity for particle aggregation in the surrounding medium. The polydispersity index (PDI) quantifies the variability in particle size within a sample, which reflects its heterogeneity. The analysis revealed that the surface of the pcMSCs-EVs displayed a negative charge, which enhanced the stability of EVs by preventing their aggregation and clumping through electrostatic repulsion. Moreover, the high PDI value suggested a considerable heterogeneity in the size of EVs within the sample, indicating the presence of a wide range of vesicle sizes (FIG. 1, (B)).

Flow cytometry analysis was conducted to examine the membrane CD markers of pcMSCs-EVs. The findings indicated that pcMSCs-EVs exhibited significant expression levels of tetraspanins CD9, CD63, and CD81 (FIG. 1, (C)), which are known to be highly concentrated in the exosomal membrane and are commonly utilized as biomarkers for the identification of exosomes.

### Example 2 In Vitro and In Vivo Effects of pcMSCs-Based Therapy on Liver Injury

### 2.1 The Survival of C3A Cells In Vitro

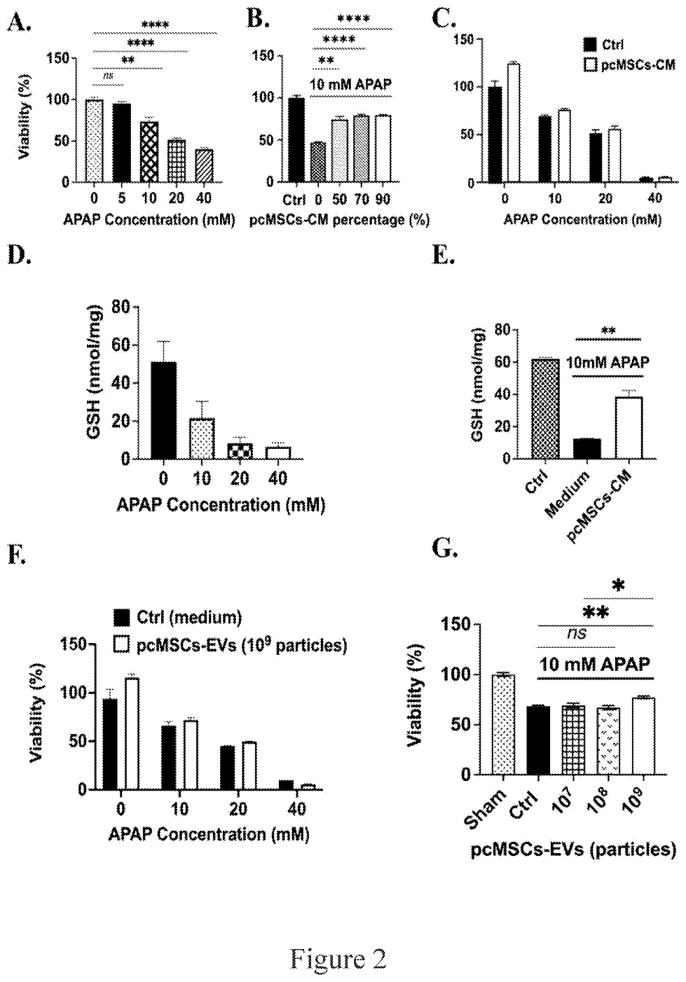

The pcMSCs-based therapies on the viability of C3A cells were investigated in this example. The viability of C3A cells was found to be dose-dependently reduced upon exposure to APAP for 24 hours (FIG. 2, (A)). However, a significant improvement in cell viability was observed after 48 hours of incubation with pcMSCs-CM in the presence of 10 mM and APAP (FIG. 2, (B)). Besides, pcMSCs-CM also enhanced the cell viability percentages of C3A cells when exposed to different concentrations of APAP (FIG. 2, (C)). Furthermore, APAP incubation resulted in a dose-dependent reduction in the expression of glutathione (GSH), which is a crucial antioxidant molecule that provides protection against oxidative stress, actively participates in the detoxification of xenobiotics (FIG. 2, (D)). Interestingly, GSH levels exhibited a significant increase after 48 hours of incubation with pcMSCs-CM in the presence of 10 mM APAP (FIG. 2, (E)). Furthermore, in order to investigate the therapeutic effects of the MSCs' secretome in the cultured medium, we purified pcMSCs-EVs in the cultured medium and administered them to injured C3A cells. The results demonstrated that the pcMSCs-EVs have the ability to enhance the survival of C3A cells in a dose-dependent manner when exposed to 10 mM APAP incubation (FIG. 2, (F)). In addition, pcMSCs-EVs also enhanced the cell viability percentages of C3A cells when exposed to different concentrations of APAP (FIG. 2, (G)). These findings highlight the potential of pcMSCs-based therapies to protect C3A cells from APAP-induced injury in an in vitro setting. The significant improvement in cell viability and the elevation of GSH levels suggested that pcMSCs-CM and pcMSCs-EVs of Example 1 may exert beneficial effects by enhancing cellular antioxidant capacity.

2.2 ALF-mouse model

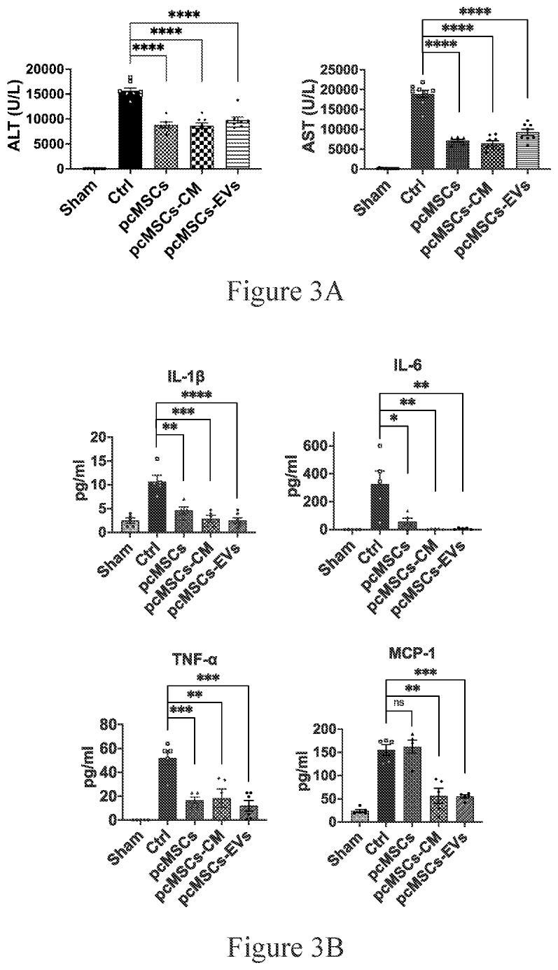

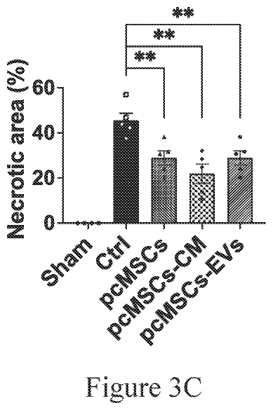

The pcMSCs-based therapy in alleviating APAP-induced acute liver failure (ALF) was evaluated by an ALF mouse model established by procedures described in the “Materials and Methods” section. Briefly, male C57BL/6 mice, aged 8-12 weeks, were subjected to a fasting period of 12-16 hours before receiving an intraperitoneal injection of 250 mg/kg body weight APAP. Then, at 30 minutes following APAP injection, the mice received either pcMSCs transplantation (1×10⁷), pcMSCs-conditioned medium (pcMSCs-CM) administration (1 mL), or pcMSCs-derived extracellular vesicles (pcMSCs-EVs) of Example 1 administration (10⁹ particles). After 24 hours, the mice were sacrificed, and blood and liver samples were collected for morphology and pathology analyses. Results are provided in FIGS. 3A to 3C.

The liver morphology of the sham group appeared smooth, exhibiting a dense texture and a brown color; conversely, the livers of the pathological control group (APAP/PBS) displayed a severe hemorrhage phenomenon. Further, the liver samples collected from mice that received pcMSCs, pcMSCs-CM or pcMSCs-EVs treatments exhibited reduced bleeding and a smoother appearance in comparison to the control livers, resembling the characteristics observed in the sham group (data not shown).

Serum levels of alanine aminotransferase (ALT) and aspartate aminotransferase (AST) were also measured to assess liver function. The results demonstrated that transplantation of pcMSCs, administration of pcMSCs-CM, and pcMSCs-EVs significantly reduced the levels of ALT and AST compared to the pathological control group (APAP/PBS) (FIG. 3A). This indicated that pcMSCs, pcMSCs-CM, or pcMSCs-EVs could provide a beneficial effect on reducing liver damage, as evidenced by the improved liver enzyme levels. Moreover, the serum levels of pro-inflammatory cytokines interleukin-1 beta (IL-1β), interleukin-6 (IL-6), and tumor necrosis factor-alpha (TNF-α), known to contribute to liver inflammation, were reduced following the administration of pcMSCs, pcMSCs-CM, or pcMSCs-EVs. Additionally, the level of monocyte chemoattractant protein-1 (MCP-1), which plays a role in immune modulation and recruitment of immune cells, was also decreased following the administration of pcMSCs, pcMSCs-CM, or pcMSCs-EVs (FIG. 3B).

These findings collectively suggest a favorable response to the intervention, characterized by improved liver health and reduced inflammation. Histological analysis using hematoxylin and eosin (H&E) staining of liver sections demonstrated that pcMSCs transplantation and pcMSCs-CM, pcMSCs-EVs administration effectively reduced the necrotic areas of liver tissue compared to the pathological control group (APAP/PBS) (FIG. 3C). Immunohistochemistry (IHC) confirmed that the expression of proliferating cell nuclear antigen (PCNA), a pivotal protein involved in DNA replication and repair in eukaryotic cells, increased in hepatocytes following pcMSCs transplantation (data not shown).

The results indicated that pcMSCs-based therapies, including transplantation and administration of pcMSCs, pcMSCs-CM, or pcMSCs-EVs of Example 1, exerted therapeutic effects in the APAP-induced ALF model. These effects were demonstrated by improvements in liver morphology, reduction in liver necrosis, and attenuation of liver injury markers and inflammatory cytokine levels, exhibiting characteristics of anti-inflammatory activity of pcMSCs.

2.3 The Biodistribution of pcMSCs-EVs

To investigate the biodistribution of pcMSCs-EVs, the membranes of pcMSCs-EVs were labeled with DiR, a near-infrared fluorescent dye suitable for in vivo imaging. The results revealed that DiR-labeled pcMSCs-EVs showed a preference for accumulation in the liver and spleen after 24 hours of peritoneal administration, compared to the 2-hour group (data not shown). Quantitative analysis indicated that DiR-labeled pcMSCs-EVs tended to be absorbed by the liver and spleen, which are major components of the reticuloendothelial system responsible for engulfing and removing foreign substances, including pathogens, cellular debris, and foreign particles, from the bloodstream and tissues.

### Example 3 Production and Characterization of Targeting EVs (TG-EVs)

3.1 Labeling pcMSCs With Azido Glycans

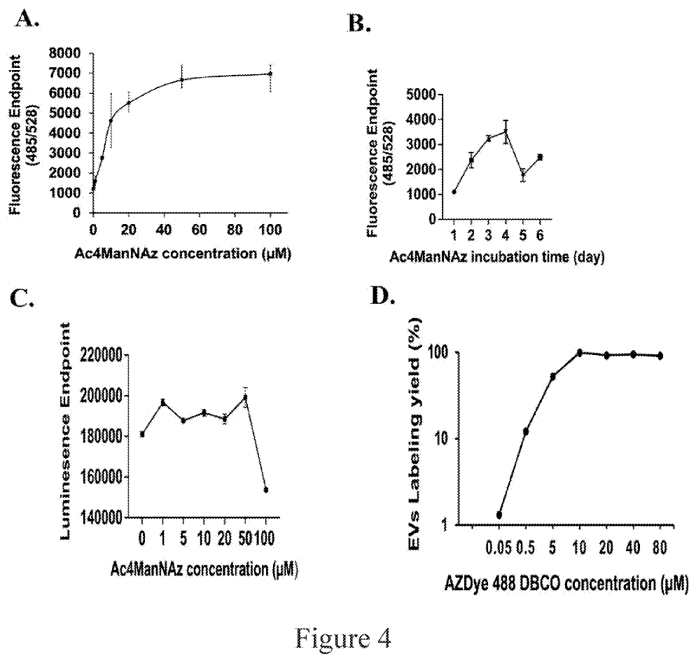

pcMSCs were incubated with increasing concentrations of Ac4ManNAz for 3 days, and the presence of the azide groups on the surface of pcMSCs was confirmed via click reactions with DBCO-fluorophore (FIG. 4, (A)). The highest level of azide groups expressed on the pcMSCs' surface was observed after 4 days of incubation with 20 μm Ac4ManNAz (FIG. 4, (B)). Furthermore, the viability of pcMSCs treated with Ac4ManNAz was unaffected at concentrations below 50 μm of Ac4ManNAz (FIG. 4, (C))

3.2 pcMSCs-EVs Having Azide Groups Expressed Thereon

Recent studies have shown that EVs secreted from azido-labeled cells also exhibit the azide groups on their surface through the exosome biogenesis pathway. Accordingly, EVs were isolated from pcMSCs treated with Ac4ManNAz as described previously, and the distribution of azide groups on the surface of EVs could be determined by performing click reactions with increasing concentrations of AZDye 488 DBCO (i.e., 0.005, 0.05, 0.5, 5, 10, 20, 40, and 80 μM), a green-fluorescent probe used for the copper-free detection of azide groups. The results showed that pcMSCs-EVs presenting azido-groups on the membrane were successfully labeled with AZDye 488

DBCO, and the labeling percentage increased dose-dependently with elevating concentrations of AZDye 488 DBCO with the maximum labeling appeared at 10 μM of AZDye 488 DBCO (FIG. 4, (D)). Additionally, images of azido-expressing pcMSCs labeled with DBCO-Cy5 through click reactions were obtained, and the quantitated results showed that pcMSCs demonstrated intense red fluorescence after click reaction with DBCO-Cy5 (data not shown).

Taken together, these findings confirmed that pcMSCs can be successfully labeled with azido groups thereby conferring EVs derived therefrom to comprise azido groups on their outer surfaces as well.

3.3 Production and Characterization of scFv Targeting Asialoglycoprotein Receptor 1 (ASGR1)

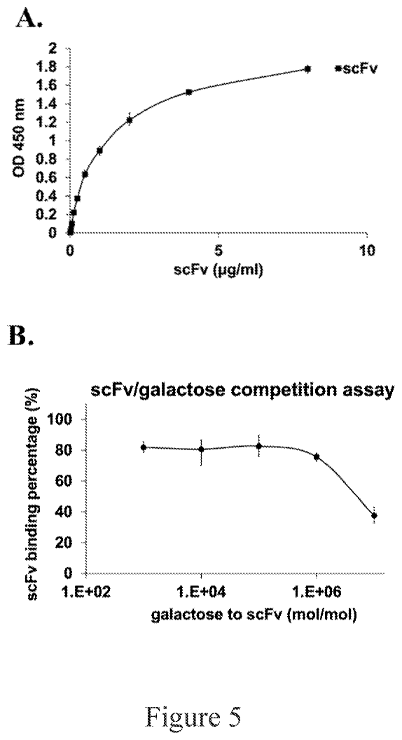

To achieve liver targeting, a specifically designed single-chain variable fragment (scFv) designed to selectively identify ASGR1, a receptor specifically present in liver cells, was developed. The recombinant scFv (SEQ ID NO:1) was composed of the variable regions from both the heavy and light chains of an antibody, joined together by a peptide linker. The C-terminus of the scFv was modified with a cysteine mutation and a His-tag. The cysteine mutation was designed to react with DBCO-PEG4-Maliemide by a site-specific cyclooctyne reaction to form a thioester bond. To verify the targeting efficacy of the scFv, sandwich ELISA and internalization experiments were conducted. The results are depicted in FIG. 5.

The results showed that the synthesized scFv specifically targets ASGR1 in a dose-dependent manner, in which the binding efficacy of the scFv increased gradually until it reached a plateau at concentrations exceeding 1 μg/ml of scFv (FIG. 5, (A)). Moreover, scFv targeted HepG2 cells at 30 mins of incubation and was gradually internalized into the cytoplasm with prolonged incubation time (1 h, 3 h, 6 h) (data not shown). To confirm that the scFv specifically binds to ASGR1 of the cells, a competition assay was conducted. Galactose, a ligand that can be specifically recognized by ASGR1, was utilized as a competitor to scFv. Various molar fractions of galactose were mixed with scFv and added to wells containing ASGR1 antigen. As the concentration of galactose increased, the signal of scFv exhibited a significant inhibition (FIG. 5, (B)). The scFv specifically targeted ASGR1 expressed on the membrane of HepG2 cells. This targeting effect was blocked by pre-incubating with galactose for one hour (data not shown).

3.4 Production scFv-DBCO

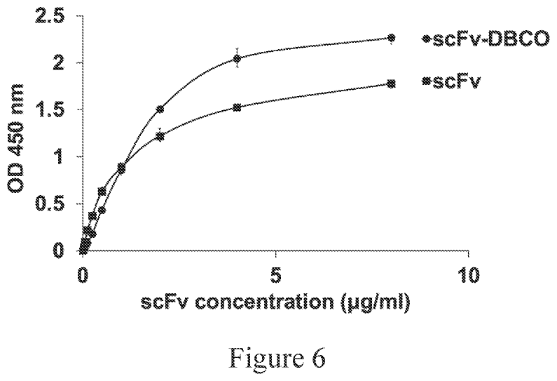

To achieve the conjugation of the scFv of Example 3.3 to DBCO, a site-specific cysteine-cyclooctyne reaction was performed. A bifunctional cross-linker called DBCO-PEG4-Maleimide, which incorporated a maleimide and an aza-dibenzocyclooctyne (DBCO), was employed to link the cysteine residue in scFv of Example 3.3 and azide on the membrane of pcMSCs-EVs respectively. This reaction involves the reaction of the cysteine residue in the C-terminus of scFv with maleimide, resulting in the formation of a thioester bond between a sulfhydryl group and a maleimide group. This conjugation leads to the conjugation of the scFv of Example 3.3 to the DBCO-PEG4-Maleimide, forming “scFv-DBCO”. The SDS-PAGE analysis demonstrated that scFv (25kDa) was successfully conjugated to DBCO-PEG4-Maleimide in a dose-dependent manner (data not shown), and the conjugation was confirmed through the detection of scFv-DBCO using CalFluor 488 Azide as a reporter. Furthermore, a sandwich ELISA was conducted to assess the binding efficiency of the following DBCO modification, and the results are depicted in FIG. 6. The results indicated that the scFv-DBCO maintained a promising binding efficiency to ASGR1, and this binding was found to be dose-dependent. Thus, the DBCO modification did not compromise the scFv's ability to effectively bind to its target receptor, ASGR1.

In this example, a highly specific targeting chemical material called scFv-DBCO was developed, which could be utilized for the surface modification of N3-pcMSCs-EVs, thereby creating a novel therapeutic platform termed “TG-EVs”, which are described in the following examples.

### 3.5 Production and Characterization of TG-EVs

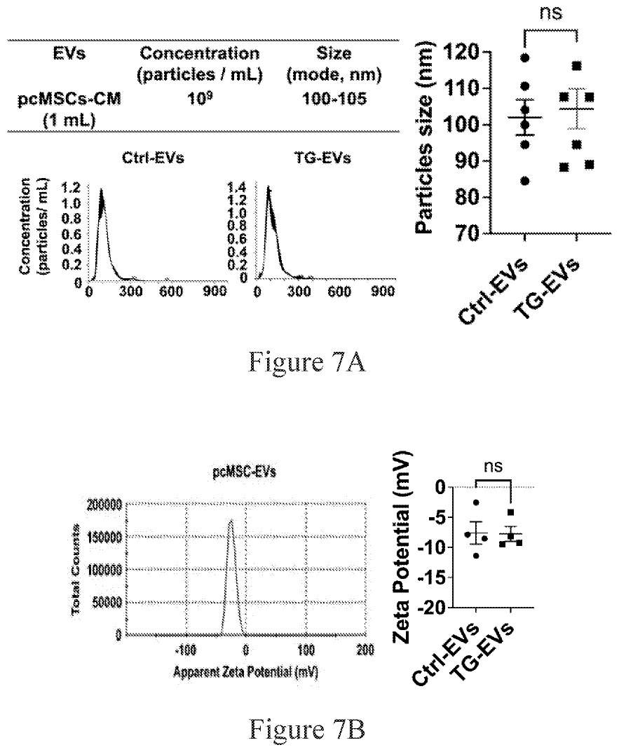

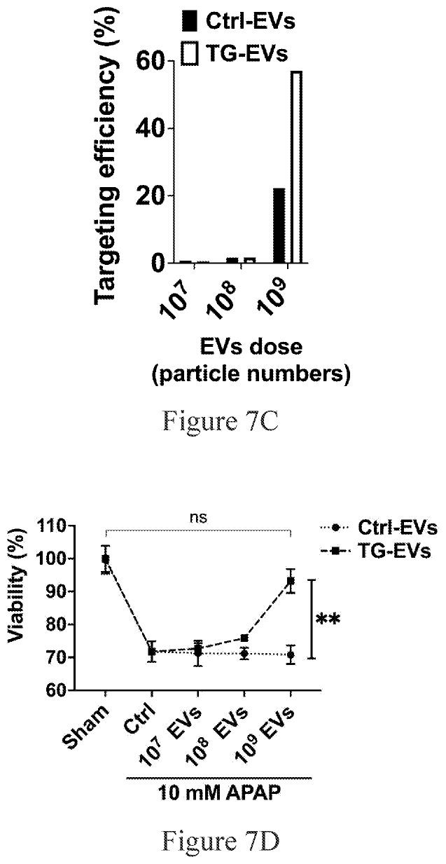

The pcMSCs-EVs of Example 3.2 and the scFv-DBCO of Example 3.4 were conjugated together via click chemistry to produce the desired TG-EVs (i.e., pcMSCs-EVs having the ASGR1-specific scFv conjugated thereto via click reaction) in accordance with procedures described in “Materials and methods” section. The thus produced TG-EVs were then subjected to morphology, size, flow cytometry, and targeting efficiency analyses. The results are depicted in FIG. 7.

TEM analysis revealed that the TG-EVs also exhibited a round or cup-shaped morphology similar to that of pcMSC-EVs (i.e., Crtl-EVs), with diameters ranging from 30 to 150 nanometers (data not shown). Nanoparticle tracking analysis (NTA) confirmed that both the Crtl-EVs and TG-EVs were about 100 to 150 nm in size, with particle concentrations of roughly 10⁹ particles/ml (FIG. 7, (A)). The analysis of the zeta potential (ZP) of both Crtl-EVs and TG-Evs revealed that the surface of both Crtl-Evs and TG-Evs displayed a negative charge (FIG. 7, (B)), which enhanced the stability of Evs by preventing their aggregation and clumping through electrostatic repulsion. Flow cytometry analysis indicated that both Crtl-Evs and TG-Evs exhibited significant expression levels of tetraspanins CD9, CD63, and CD81 (data not shown). As to targeting efficiency, HepG2/C3A cells incubated with increasing doses (10⁷, 10⁸, 10⁹) of TG-EVs were found to exhibit significant Alexa Fluor-488 and Cy5 fluorescent signal in a dose-dependent manner as compared to that of Crtl-EVs (FIG. 7, (C)), an indication that the TG-EVs could successfully bind to ASGR1 on the membranes of HepG2/C3A cells.

For therapeutic efficiency, TG-EVs significantly improved the viability of HepG2/C3A cells dose-dependently compared to corresponding doses of Ctrl-EVs after 48 hours of incubation in the presence of APAP (10 mM) (FIG. 7, (D)). These findings highlight the potential of TG-EVs to protect the liver from APAP-induced injury in an in vitro setting.

### Example 4 TG-EVs Exhibited Superior Therapeutic Effect in Attenuating APAP-Induced Liver Injury in the ALF-Mouse Model

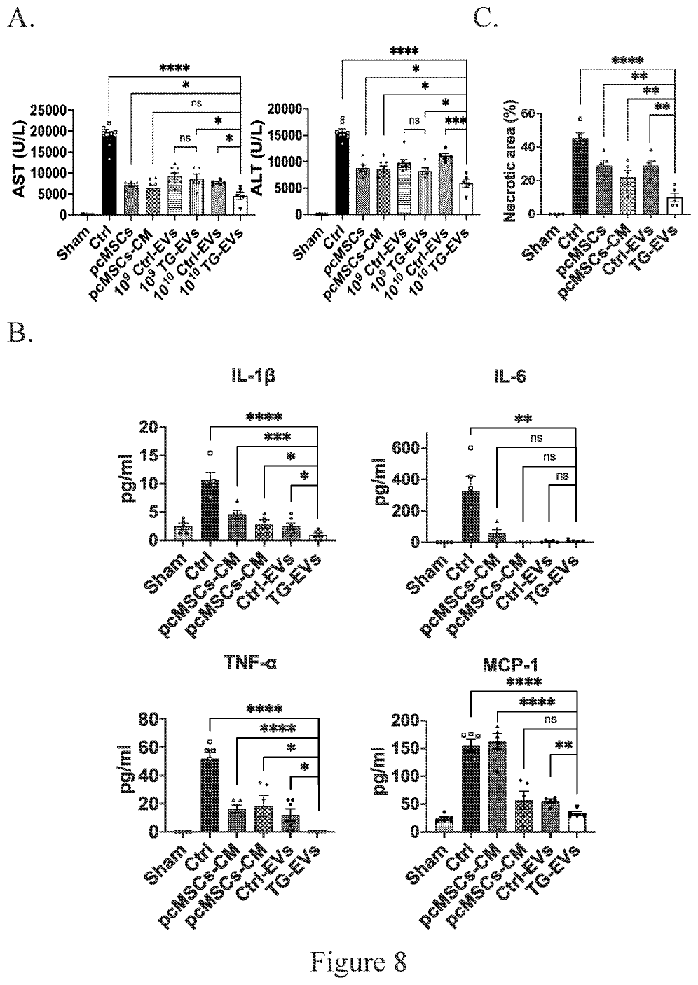

In this example, the efficiency of TG-EVs of Example 3.5 in APAP-induced liver injury was evaluated in the ALF-mouse model. Briefly, after fasting for 12-16 hours, male C57BL/6 mice (8-12 weeks old) mice received one injection of APAP (250 mg/kg) intraperitoneally and subsequently received pcMSCs (1×10⁷ cells) transplantation, pcMSC-CM (1 mL), Ctrl-EVs or TG-EVs (10⁹, 10¹⁰ particles) administration at 30 min following APAP injection. Mice were then sacrificed, and the blood and livers were collected 24 hours after APAP injection. Serum levels of ALT, AST, and inflammatory cytokines, as well as necrotic area were determined, and results are provided in FIG. 8.

Similar to findings in Example 2.2, data in this example also indicated that pcMSCs, pcMSCs-CM, Ctrl-EV, and TG-EVs could provide a beneficial effect on reducing liver damage, as evidenced by the improved ALT and AST levels (FIG. 8, (A)). Moreover, the serum levels of pro-inflammatory cytokines IL-1β, IL-6, and TNF-α, as well as the level of MCP-1 were reduced following the administration of pcMSCs, pcMSCs-CM, Ctrl-EV, or TG-EVs (FIG. 8, (B)). Additionally, the necrotic area also decreased following the administration of pcMSCs, pcMSCs-CM, Ctrl-EV, or TG-EVs, as compared to that of the untreated mice (FIG. 8, (C)).

Taken together, the present disclosure provides a novel platform for the production of targeting EVs, which can specifically recognize and bind to desired sites in a subject, these targeting EVs are therefore candidates for the development of medicaments for the treatment of diseases, such as ALF as demonstrated in this application.

It will be understood that the above description of embodiments is given by way of example only and that various modifications may be made by those with ordinary skill in the art. The above specifications, examples, and data provide a complete description of the structure and use of exemplary embodiments of the invention. Although various embodiments of the invention have been described above with a certain degree of particularity, or regarding one or more individual embodiments, those with ordinary skill in the art could make numerous alterations to the disclosed embodiments without departing from the spirit or scope of this invention.

What is claimed is:

1. An extracellular vesicle (EV) composite comprising an EV derived from a mesenchymal stem cell (MSC), and a recombinant polypeptide of SEQ ID No: 1 conjugated to the EV via a click chemistry reaction.

2. The EV composite of claim 1, wherein the EV is derived from the MSC treated with N-azidoacetyl-mannosamine thereby conferring the EV to comprise an azide on its outer surface, while the recombinant polypeptide is treated with a molecule having a click chemistry group that is dibenzocyclooctyne (DBCO), difluorinated cyclooctyne (DIFO), biarylazacyclooctynone (BARAC) or bicyclononyne (BCN) thereby conferring the recombinant polypeptide to comprise a strained alkyne, and the recombinant polypeptide is conjugated to the EV via strain promoted azide-alkyne cycloaddition (SPAAC).

3. The EV composite of claim 2, wherein the click chemistry group is DBCO.

4. The EV composite of claim 1, wherein the EV is derived from the MSC treated with 1,2,3,4-tetrazine, 1,2,3,5-tetrazine, or 1,2,4,5-tetrazine thereby conferring the EV to comprise a tetrazine on its outer surface, while the recombinant polypeptide is treated with a molecule having a click chemistry group that is oxanorbornadiene or cyclooctene thereby conferring the recombinant polypeptide to comprise an alkene, and the recombinant polypeptide is conjugated to the EV via the alkene and tetrazine inverse-demand Diels-Alder reaction.

5. The EV composite of claim 1, the MSC is selected from the group consisting of adipose-derived MSC (AD-MSC), amniotic fluid-derived MSC (AF-MSC), bone marrow-derived MSC (BM-MSC), dental pulp-derived MSC (DP-MSC), endometrial-derived MSC (En-MSC), fallopian tube-derived MSC (FT-MSC), gingival-derived MSC (G-MSC), periodontal ligament-derived MSC (PDL-MSC), peripheral blood-derived MSC (PB-MSC), placenta-derived MSC (PD-MSC), placenta choriodecidua-derived MSC (PC-MSC), skeletal muscle-derived MSC (Sk-MSC), synovial membrane-derived MSC (SM-MSC), umbilical cord-derived MSC (UCB-MSC), and Wharton's Jelly-derived MSC (WJ-MSC).

6. The EV composite of claim 5, wherein the MSC is PC-MSC.

7. A method of treating acute liver failure (ALF) in a subject comprising administering an effective amount of an extracellular vesicle (EV) composite to the subject to alleviate symptoms associated with the ALF, wherein the EV composite comprises an EV derived from a mesenchymal stem cell (MSC) and a recombinant polypeptide of SEQ ID Nos: 1 or 2 conjugated to the EV via a click chemistry reaction.

8. The method of claim 7, wherein the EV composite is administered to the subject in the amount of about 1×10⁸ to 1×10¹² EV composite/mL.

9. The method of claim 7, wherein the EV is derived from the MSC treated with N-azidoacetyl-mannosamine thereby conferring the EV to comprise an azide on its outer surface, while the recombinant polypeptide is treated with a molecule having a click chemistry group that is dibenzocyclooctyne (DBCO), difluorinated cyclooctyne (DIFO), biarylazacyclooctynone (BARAC) or bicyclononyne (BCN) thereby conferring the recombinant polypeptide to comprise a strained alkyne, and the recombinant polypeptide is conjugated to the EV via strain promoted azide-alkyne cycloaddition (SPAAC).

10. The method of claim 9, wherein the click chemistry group is DBCO.

11. The method of claim 7, wherein the EV is derived from the MSC treated with 1,2,3,4-tetrazine, 1,2,3,5-tetrazine, or 1,2,4,5-tetrazine thereby conferring the EV to comprise a tetrazine on its outer surface, while the recombinant polypeptide is treated with a molecule having a click chemistry group that is oxanorbornadiene or cyclooctene thereby conferring the recombinant polypeptide to comprise an alkene, and the recombinant polypeptide is conjugated to the EV via alkene and tetrazine Inverse-demand Diels-Alder reaction.

12. The method of claim 7, the MSC is selected from the group consisting of adipose-derived MSC (AD-MSC), amniotic fluid-derived MSC (AF-MSC), bone marrow-derived MSC (BM-MSC), dental pulp-derived MSC (DP-MSC), endometrial-derived MSC (En-MSC), fallopian tube-derived MSC (FT-MSC), gingival-derived MSC (G-MSC), periodontal ligament-derived MSC (PDL-MSC), peripheral blood-derived MSC (PB-MSC), placenta-derived MSC (PD-MSC), placenta choriodecidua-derived MSC (PC-MSC), skeletal muscle-derived MSC (Sk-MSC), synovial membrane-derived MSC (SM-MSC), umbilical cord-derived MSC (UCB-MSC), and Wharton's Jelly-derived MSC (WJ-MSC).

13. The method of claim 12, wherein the MSC is PC-MSC.

14. The method of claim 7, further comprising administering to the subject a therapeutic agent selected from the group consisting of interferon, adefovir, entecavir, lamivudine, telbivudine, tenofovir, and silymarin.

15. The method of claim 7, wherein the subject is a human.


SEQUENCE LISTING (1 sequence)

SEQ ID NO: 1 - protein, 268 aa, synthetic construct
MAEVQLLESGGGLVQPGGCLRLSCAASGFTFSDYYMSWVRQAPGKGLEWVSAITTGGGSPNYADSVKGRFTISRDNSKNTLYLQMNSLRAEDTAVYYCARDRTAGYFDYWGQGALVTVSSGSAGGGGSGGGGSGGGGSDIVLTQPPSASGTPGQRVTISCTGSSSGIGNAYVSWYQQLPGKAPKLLIYKNGQRPSGVSDRFSGSKSGTSASLAISGLRSEDEADYYCAAWDDSLNGWVFGGGTKVTVLRGGGLPETGGGSHHHHHHHH